(19) United States
(12) Patent Application Publication
(10) Pub. No.: US 2011/0282081 A1
(43) Pub. Date: Nov. 17, 2011

(54) Methods of Preparing 1-Deoxy-Sphingoid Bases and Derivatives Thereof

(75) Inventors: Lanny S. Liebeskind, Atlanta, GA (US); Ethel C. Garnier-Amblard, Decatur, GA (US); Dennis C. Liotta, Atlanta, GA (US)

(73) Assignee: EMORY UNIVERSITY, Atlanta, GA (US)

(21) Appl. No.: 13/143,611

(22) PCT Filed: Jan. 21, 2010
(86) PCT No.: PCT/US10/21624

§ 371 Date: Aug. 2, 2011

(51) Int. Cl. C07D 301/14; C07C 213/08; C07F 7/18

(52) U.S. Cl. 549/523; 556/418; 564/468; 564/507; 564/503; 564/487

(57) ABSTRACT

Novel methods of synthesizing 1-deoxy-sphingoid bases and derivatives are disclosed. The synthesis is achieved from commercially available and inexpensive starting materials. The process includes thioesterification, cross-coupling, and reduction. The process may also include directed epoxidation, regioselective epoxide-opening, hydrogenation, and dihydroxylation. The methods described herein provide 1-deoxy-sphingoid bases and derivatives in high overall yield and high enantiomeric purity.

### CROSS-REFERENCE TO PRIORITY APPLICATION

This application claims priority to U.S. Provisional Application No. 61/146,925, filed Jan. 23, 2009, which is incorporated herein by reference in its entirety.

This invention was made with government support under Grant No. GM 066153 awarded by the National Institute of Health. The government has certain rights in the invention.

### BACKGROUND

Enigmol, a synthetic sphingolipid analogue, suppresses the growth of several cancer cell lines, including those of the brain, blood, breast, colon, ovary, pancreas, and prostate. In addition, enigmol is more potent than other sphingolipids and also possesses good pharmacokinetic properties, including oral bioavailability. Enigmol belongs to a class of compounds referred to as 1-deoxy-sphingoid bases, a group that contains several compounds with potent anti-cancer activity. For example, the natural product spisulosine is an investigational anti-cancer drug that has been shown to be effective in numerous cancer cell lines. Other examples of 1-deoxy-sphingoid bases that display cytoxicity in cancer cell lines include the obscuraminols and the crucigasterins. Thus, the 1-deoxy-sphingoid bases and derivatives have potential as therapeutic treatments for a number of cancer types. However, current methods of synthesizing compounds from this class are impractical due to poor product yield and low enantiopurity.

### SUMMARY

Novel methods of preparing 1-deoxy-sphingoid bases and derivatives are provided. The methods include making a compound of formula (I):

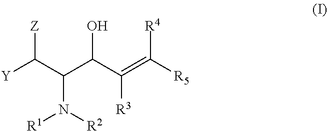

and pharmaceutically acceptable salts and prodrugs thereof, wherein R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl; R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl; Y is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, substituted or unsubstituted heteroaryl, or substituted or unsubstituted carboxyl; and Z is H, OH, SH, NR¹R², or substituted or unsubstituted alkyl, with the proviso that when Z is OH, Y is not H. The method of making the compound of formula (I) comprises:

(a) converting a compound of formula (II):

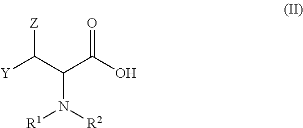

to a compound of formula (III):

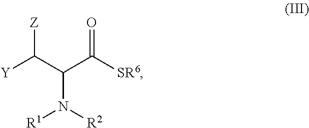

wherein R⁶ is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;

(b) cross-coupling the compound of formula (III) with a compound of the following structure:

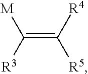

wherein M is B(R⁷)₂, Sn(R⁷)₃, Si(R⁷)₃, ZnR⁷, or InR⁷R⁸, wherein R⁷ and R⁸ are each independently selected from halogen, substituted or unsubstituted alkyl, substituted or unsubstituted alkenyl, substituted or unsubstituted alkynyl, substituted or unsubstituted aryl, substituted or unsubstituted alkoxy, substituted or unsubstituted hydroxyl, substituted or unsubstituted heteroalkyl, substituted or unsubstituted heteroalkenyl, substituted or unsubstituted heteroalkynyl, or substituted or unsubstituted heteroaryl;

to form a compound of formula (IV):

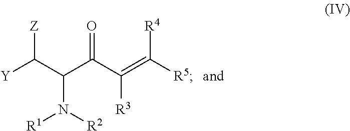

(c) reducing the compound of formula (IV) with a reducing agent to form a compound of formula (I). In some embodiments, the compound of formula (II) has the following structure:

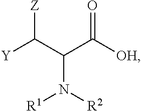

wherein R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl; Y is substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, substituted or unsubstituted heteroaryl, or substituted or unsubstituted carboxyl; and Z is H, OH, SH, NR¹R², or substituted or unsubstituted alkyl.

Also provided is a method of making a compound of formula (V):

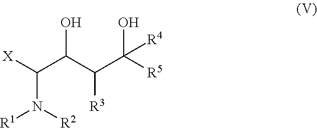

and pharmaceutically acceptable salts and prodrugs thereof, wherein R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl; R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl; and X is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl. The method of making compound (V) comprises:

(a) converting a compound of formula (VI):

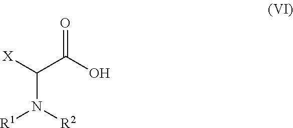

to a compound of formula (VII):

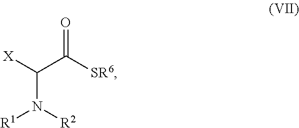

wherein R⁶ is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;

(b) cross-coupling the compound of formula (VII) with a compound of the following formula:

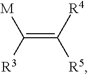

wherein M is B(R⁷)₂, Sn(R⁷)₃, Si(R⁷)₃, ZnR⁷, or InR⁷R⁸, wherein R⁷ and R⁸ are each independently selected from halogen, substituted or unsubstituted alkyl, substituted or unsubstituted alkenyl, substituted or unsubstituted alkynyl, substituted or unsubstituted aryl, substituted or unsubstituted alkoxy, substituted or unsubstituted hydroxyl, substituted or unsubstituted heteroalkyl, substituted or unsubstituted heteroalkenyl, substituted or unsubstituted heteroalkynyl, or substituted or unsubstituted heteroaryl;

to form a compound of formula (VIII):

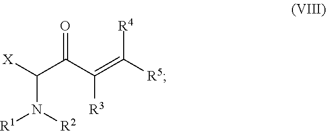

(c) reducing the compound of formula (VIII) with a reducing agent to form a compound of formula (IX):

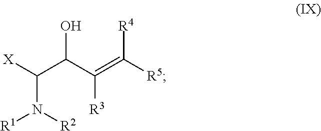

(d) performing a directed epoxidation on the compound of formula (IX) to form a compound of formula (X):

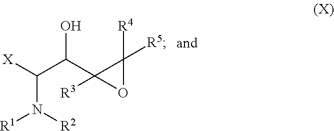

(e) opening the epoxide ring of the compound of formula (X) to form a compound of formula (V).

The details of one or more embodiments are set forth in the description below. Other features, objects, and advantages will be apparent from the description and from the claims.

### DETAILED DESCRIPTION

The term “comprising” and variations thereof as used herein are used synonymously with the term “including” and variations thereof and are open, non-limiting terms.

Novel methods of synthesizing 1-deoxy-sphingoid bases and its derivatives are disclosed. The term “sphingoid base”, as used herein, includes long carbon chain amines containing at least one hydroxyl group. The carbon chain is optionally substituted, and may be saturated or unsaturated. The compounds useful in the methods described herein include 1-deoxy-sphingoid bases. The 1-deoxy-sphingoid bases are structural analogues of sphingoid bases that lack the hydroxyl group on position 1 (i.e., the sphingobases do not contain a terminal hydroxyl group in the position adjacent to the amine). An example of a 1-deoxy-sphingoid base is enigmol:

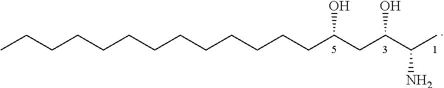

The 1-deoxy-sphingoid bases include compounds represented by formula (I):

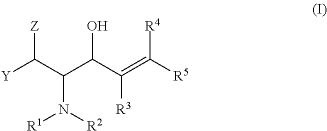

and pharmaceutically acceptable salts and prodrugs thereof. In formula (I), R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl; R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl; Y is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, substituted or unsubstituted heteroaryl, or substituted or unsubstituted carboxyl; and Z is H, OH, SH, NR¹R², or substituted or unsubstituted alkyl, with the proviso that when Z is OH, Y is not H.

As used herein, the terms “alkyl”, “alkenyl”, and “alkynyl” can include straight-chain, branched, and cyclic monovalent substituents. Examples include methyl, ethyl, isobutyl, cyclohexyl, cyclopentylethyl, 2-propenyl, 3-butynyl, and the like. “Heteroalkyl”, “heteroalkenyl”, and “heteroalkynyl” refer to compounds that are similar to “alkyl”, “alkenyl”, and “alkynyl” but that further contain a hetero atom such as O, S, or N or combinations thereof.

As used herein, the term “aryl” can include monocyclic or fused bicyclic moieties such as phenyl or naphthyl and the term “heteroaryl” can include monocyclic or fused bicyclic ring systems containing one or more heteroatoms selected from O, S, and N. Heteroaryls can include, for example, 5-, 6-, 7-, and 8-membered rings. Thus, aryl and heteroaryl systems can include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl, and the like.

As used herein, the term “substituted” indicates the main substituent has attached to it one or more additional components, such as, for example, OH, halogen, or one of the other substituents listed above. Substituted aryls can include, for example, monosubstituted, disubstituted, or trisubstituted aryls.

The method of making the compounds of formula (I) includes the steps detailed herein. In step (a), an acid is converted to the corresponding thiol ester in a thioesterification reaction. In some embodiments, a compound of formula (II):

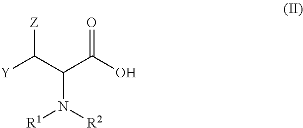

is converted to a compound of formula (III):

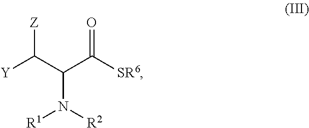

wherein R⁶ is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl.

In some embodiments, the compound of formula (II) has the following structure:

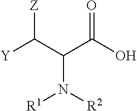

wherein R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl; Y is substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, substituted or unsubstituted heteroaryl, or substituted or unsubstituted carboxyl; and Z is H, OH, SH, NR¹R², or substituted or unsubstituted alkyl. In some embodiments, the compound of formula (II) is an amino acid. For example, the compound of formula (II) can be threonine, cysteine, asparagine, or proline.

In some embodiments, the acid is a compound of formula (VI):

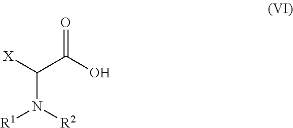

and the thiol ester is a compound of formula (VII):

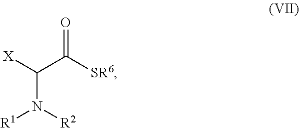

wherein R⁶ is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl.

In some embodiments, R¹ or R² is hydrogen, acetyl, alkyl acetyl, benzyl, t-butoxycarbonyl (Boc), benzyloxycarbonyl (Cbz), or 9-fluorenylmethoxycarbonyl (Fmoc). In some embodiments, the compound of formula (VI) is an N-protected amino acid. For example, the compound of formula (VI) can be N-Boc-L-alanine or N-Boc-D-alanine, wherein X is methyl, R¹ is H, and R² is Boc.

Formation of a compound of formula (III) or formula (VII) can be accomplished by a peptidic coupling reaction. For example, the compound of formula (II) or formula (VI) can be treated with thiophenol (PhSH), N,N-dicyclohexylcarbodiimide (DCC), and 1-hydroxybenzotriazole (HOBt) in a solvent to form the compound of formula (III) or formula (VII) wherein R⁶ is phenyl. The solvent used for the reaction can include a polar, non-protic solvent. For example, the solvent can include ethyl acetate (EtOAc), dichloromethane (CH₂Cl₂), acetonitrile (CH₃CN), dimethylformamide (DMF), or combinations thereof. The thioester of formula (III) or formula (VII) can be formed in high yield with retention of the enantiopurity.

As used herein, “enantiopurity” is expressed in terms of “enantiomeric excess” (ee), which is expressed as a percentage and is defined as: % ee=100(major−minor)/(major+minor), wherein the term “major” refers to the more abundant enantiomer and the term “minor” refers to the less abundant enantiomer.

In step (b), a carbon-carbon bond is formed in a cross-coupling by reacting the thiol ester of formula (III) or (VII) with an organometallic compound of the following structure:

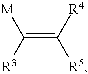

wherein M is B(R⁷)₂, Sn(R⁷)₃, Si(R⁷)₃, ZnR⁷, or InR⁷R⁸, wherein R⁷ and R⁸ are each independently selected from halogen, substituted or unsubstituted alkyl, substituted or unsubstituted alkenyl, substituted or unsubstituted alkynyl, substituted or unsubstituted aryl, substituted or unsubstituted alkoxy, substituted or unsubstituted hydroxyl, substituted or unsubstituted heteroalkyl, substituted or unsubstituted heteroalkenyl, substituted or unsubstituted heteroalkynyl, or substituted or unsubstituted heteroaryl.

In some embodiments, the compound of formula (III) is reacted with an organometallic compound to form a compound of formula (IV):

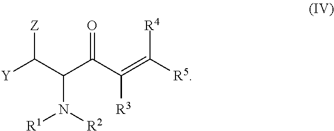

In some embodiments, the compound of formula (VII) is reacted with an organometallic compound to form a compound of formula (VIII):

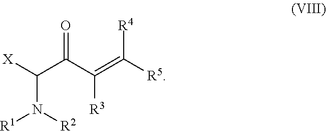

In some embodiments, the organometallic compound is an organoborane compound, i.e., M is B(R⁷)₂. For example, M can be a boronic acid, boronic ester, boron halide, or alkyl borane. In some embodiments, the organometallic compound is a boronic acid of the following structure:

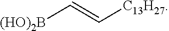

The cross-coupling in step (b) can be performed in the presence of a transition metal catalyst, a copper carboxylate, and a supporting ligand. The transition metal catalyst can include, for example, a palladium catalyst, a platinum catalyst, a rhodium catalyst, a nickel catalyst, an iridium catalyst, an iron catalyst, a copper catalyst, a cobalt catalyst, a ruthenium catalyst, and combinations thereof. In some embodiments, the transition metal catalyst is tris(dibenzylideneacetone)dipalladium(0) (Pd₂(dba)₃). The copper carboxylate can include a copper (I) carboxylate. For example, the copper carboxylate can be selected from copper (I) 2-thiophenecarboxylate (CuTC), copper (I) methyl-salicylate, copper (I) diphenylphosphinate, and combinations thereof. The supporting ligand can be selected from a phosphite ligand, a phosphine ligand, a nitrogen ligand, an antimony ligand, and combinations thereof. In some embodiments, the supporting ligand is triethyl phosphite (P(OEt)₃).

The solvent used for the reaction can include a polar, non-protic solvent. For example, the solvent can include tetrahydrofuran (THF), DMF, or combinations thereof. The cross-coupling step exhibits high yields and enantioselectivity.

In step (c), the compound of formula (IV) or formula (VIII) is reduced with a reducing agent. As used herein, the term “reducing agent” refers to a compound or combination of compounds that furnishes electrons to another compound. The term “reducing agent” includes hydride reducing agents that include a negatively charged hydrogen (e.g., lithium aluminum hydride).

In some embodiments, the compound of formula (IV) is reduced to form the compound of formula (I). In some embodiments, the compound of formula (VIII) is reduced to form a compound of formula (IX):

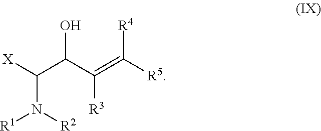

The reduction in step (c) can be performed with a number of known reducing agents, including aluminum hydrides and borohydrides. For example, the reducing agent can be selected from lithium tri-sec-butylborohydride (L-Selectride), potassium tri-sec-butylborohydride (K-Selectride), lithium tri-tert-butoxyaluminum hydride (LiAlH(Ot-Bu)₃), lithium 9-BBN hydride, lithium borohydride (LiBH₄), sodium borohydride (NaBH₄), sodium cyanoborohydride (NaBH₃CN), diisobutylaluminum hydride (DIBAL-H), and lithium aluminum hydride (LiAlH₄).

The reduction of the ketone can be performed diastereoselectively with the choice of the reducing agent. For example, the syn isomer of the compound of formula (I) formed from N-Boc-L-alanine can be synthesized using L-Selectride, while the anti isomer can be produced using LiAlH(Ot-Bu)₃. Both isomers can be synthesized in high diastereomeric ratios (i.e. greater than 95:5).

The method can also include step (d), wherein a directed epoxidation is performed on the compound. In some embodiments, a directed epoxidation is performed on the compound of formula (I) to form an α-epoxy alcohol. In some embodiments, a directed epoxidation is performed on the compound of formula (IX) to form a compound of formula (X):

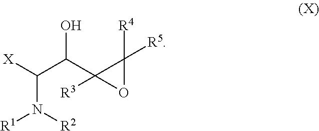

The directed epoxidation in step (d) can be performed with a number of known epoxidizing agents, including peroxyacids. For example, the epoxidizing agent can be selected from m-chloroperoxybenzoic acid (mCPBA), peroxyacetic acid, peroxybenzoic acid, trifluoroperoxyacetic acid, 3,5-dinitroperoxybenzoic acid, and tert-butylhydroperoxide on molecular sieves. In addition, the epoxidation can be performed according to the Sharpless asymmetric epoxidation conditions, by treatment with t-BuOOH, titanium tetraisopropoxide, and chiral diethyl tartrate.

The epoxidation can be performed in a stereocontrolled manner with the choice of the reagent. For example, a directed epoxidation can be performed using mCPBA or Sharpless epoxidation conditions to provide the corresponding α-epoxy alcohols in high diasteromeric ratios (i.e., greater than 95:5).

The method can additionally include step (e), wherein the epoxide ring is opened. The epoxide ring can be opened, for example, using Red-Al, to give the 1,3-diol exclusively. In some embodiments, the epoxide ring of the compound of formula (X) is opened to form a compound of formula (V):

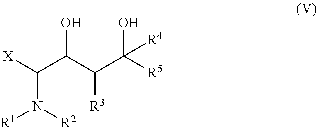

or a pharmaceutically acceptable salt or prodrug thereof.

In formula (V), R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl; R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl; and X is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl.

Alternatively, the compound of formula (I) can be hydrogenated to form the saturated version of the compound. The compound of formula (I) can also be dihydroxylated to form the diol.

In some embodiments, Z is OH. In these cases, the method further includes protecting the hydroxyl group of Z with a silyl group, an ester group, an acetal group, a carboxyl group, a glycosyl group, a phosphoryl group, or an ether group. Also, the method can further comprise deprotecting the protected alcohol group in the compound of formula (I) to form the deprotected alcohol group. Deprotection can be accomplished according to methods used by those of skill in the art and also methods found, for example, in Wuts and Greene, Protective Groups in Organic Synthesis, 4^{th} Ed., Wiley & Sons, 2006, which is incorporated herein by reference in its entirety.

Further, the method can include deprotecting the mono- or di-substituted amino group NR¹R² of the compound of formula (I) or formula (V) to form the unsubstituted amino group NH₂. The di-substituted amino group of a compound of formula (V) (i.e., NR¹R² wherein R¹ and R² are not H) can be partially deprotected to form the monosubstituted amino group (i.e., NHR¹). Deprotection can be accomplished according to methods used by those of skill in the art and also methods found, for example, in Wuts and Greene, Protective Groups in Organic Synthesis, 4^{th} Ed., Wiley & Sons, 2006, which is incorporated herein by reference in its entirety.

The compound of formula (V) containing the unsubstituted amino group (i.e., NH₂) or mono-substituted amino group (i.e., NHR¹), can be reacted with an acid or acid chloride in the presence of a condensation reagent to form the compound of formula (V) wherein R¹ is a substituted or unsubstituted carbonyl group (i.e., 1-deoxy-ceramides). For example, the compound of formula (V) containing the unsubstituted amino group (i.e., NH₂) can be reacted with the acid chloride CH₃CH₂COCl in the presence of dicyclohexylcarbodiimide to form the compound of formula (V) containing the mono-substituted amino group NHC(O)CH₂CH₃.

The reactions described herein can be performed in a stereoselective manner. Each stereoisomer of the compound of formula (V), for example, can be synthesized through the selection of the appropriate starting materials (i.e., the D or L isomer of the starting amino acid) and the selection of the reagents for the ketone reduction (i.e., L-Selectride or LiAlH(Ot-Bu)₃) and the epoxidation (i.e., mCPBA or Sharpless epoxidation conditions). For example, the following compounds can be synthesized:

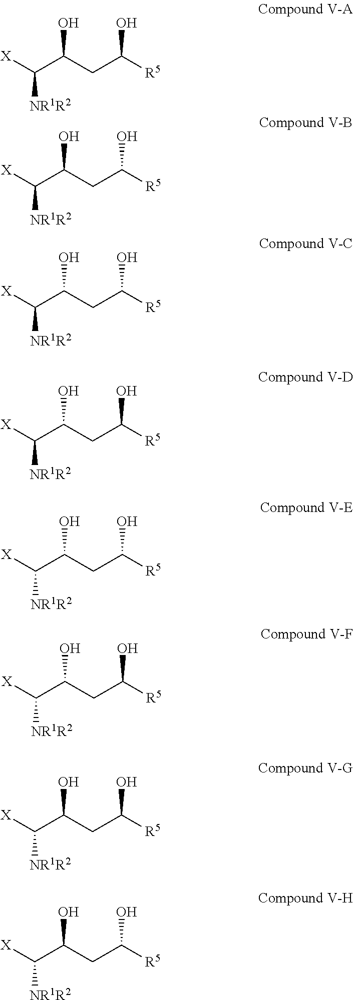

In some examples, the N-protected amino acid in step (a) can be N-Boc-L-alanine and the cross-coupling in step (b) can be performed with a compound of the following structure:

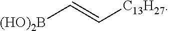

When the reducing agent in step (c) is L-Selectride, the compound of formula (IX) has the following structure:

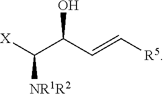

The directed epoxidation can be performed using mCPBA to form the compound of formula (X) with the following structure:

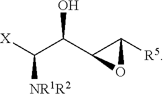

The epoxide can then be regioselectively opened in step (e) to form the compound of formula (V) with the following structure:

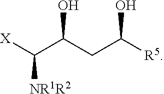

Alternatively, the directed epoxidation can also be performed using Sharpless epoxidation conditions to form the compound of formula (X) with the following structure:

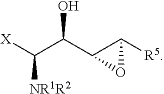

The epoxide can then be regioselectively opened in step (c) to form the compound of formula (V) with the following structure:

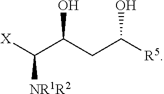

When the reducing agent in step (c) is LiAlH(Ot-Bu)₃, the compound of formula (IX) has the following structure:

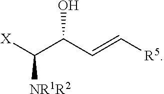

The directed epoxidation in step (d) can be performed using mCPBA to form the compound of formula (X) with the following structure:

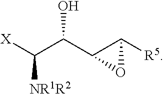

The epoxide can then be regioselectively opened in step (e) to form the compound of formula (V) with the following structure:

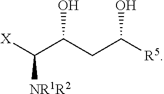

Alternatively, the directed epoxidation in step (d) can be performed under Sharpless epoxidation conditions to form the compound of formula (X) with the following structure:

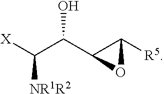

The epoxide can then be regioselectively opened in step (e) to form the compound of formula (V) with the following structure:

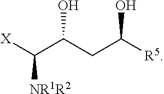

In some examples, the N-protected amino acid in step (a) can be N-Boc-D-alanine and the cross-coupling in step (b) is performed with a compound of the following structure:

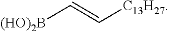

When the reducing agent in step (c) is L-Selectride, the compound of formula (IX) has the following structure:

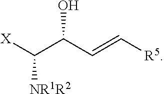

The directed epoxidation in step (d) can be performed using mCPBA to form the compound of formula (X) with the following structure:

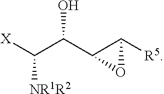

The epoxide can then be regioselectively opened in step (e) to form the compound of formula (V) with the following structure:

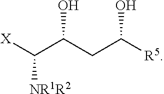

Alternatively, the directed epoxidation in step (d) can be performed under Sharpless epoxidation conditions to form the compound of formula (X) with the following structure:

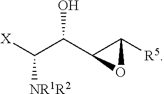

The epoxide can then be regioselectively opened in step (e) to form the compound of formula (V) with the following structure:

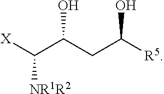

When the reducing agent in step (c) is LiAlH(Ot-Bu)₃, the compound of formula (IX) has the following structure:

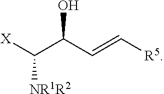

The directed epoxidation in step (d) can be performed using mCPBA to form the compound of formula (X) with the following structure:

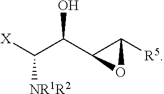

The epoxide can then be regioselectively opened in step (e) to form the compound of formula (V) with the following structure:

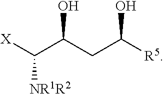

Alternatively, the directed epoxidation in step (d) can be performed under Sharpless epoxidation conditions to form the compound of formula (X) with the following structure:

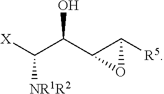

The epoxide can then be regioselectively opened in step (e) to form the compound of formula (V) with the following structure:

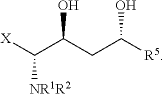

Variations on Compound I, Compound II, Compound III, Compound IV, Compound V, Compound VI, Compound VII, Compound VIII, Compound IX, and Compound X include the addition, subtraction, or movement of the various constituents as described for each compound. Similarly, when one or more chiral centers is present in a molecule the chirality of the molecule can be changed. Additionally, compound synthesis can involve the protection and deprotection of various chemical groups. The use of protection and deprotection, and the selection of appropriate protecting groups can be selected by one skilled in the art. The chemistry of protecting groups can be found, for example, in Wuts and Greene, Protective Groups in Organic Synthesis, 4^{th} Ed., Wiley & Sons, 2006, which is incorporated herein by reference in its entirety.

Reactions to produce the compounds described herein can be carried out in solvents indicated herein, or in solvents which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The examples below are intended to further illustrate certain aspects of the methods and compounds described herein, and are not intended to limit the scope of the claims.

### EXAMPLES

All reagents and solvents listed herein are commercially available (e.g., from Sigma-Aldrich; St. Louis, Mo.) unless otherwise indicated.

### Example 1

### Synthesis of Compound of Formula III

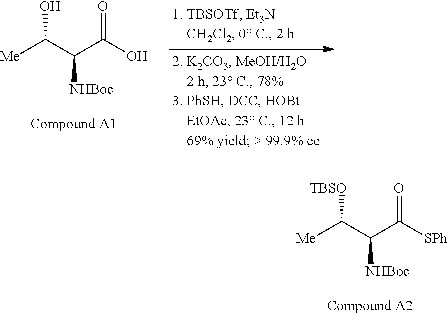

N-Boc-L-allo-threonine (Compound A1; synthesized from L-allo-threonine purchased from Fluka Chemical Corp.; Milwaukee, Wis.) was protected with TBS using tert-butyldimethylsilyl triflate (TBSOTf) and then treated with thiophenol (PhSH), N,N-dicyclohexylcarbodiimide (DCC), and 1-hydroxybenzotriazole (HOBt) in ethyl acetate (EtOAc) to form (2S,3S)—S-phenyl 2-(tert-butoxycarbonylamino)-3-(tert-butyldimethylsilyloxy)butanethioate (Compound A2). Compound A2 was formed in 69% yield and an enantiomeric excess (cc) of >99.9%.

### Example 2

### Synthesis of Compound of Formula IV

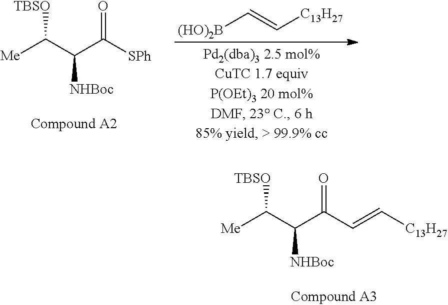

Compound A2 was reacted with (E)-pentadec-1-enylboronic acid in the presence of tris(dibenzylideneacetone)dipalladium(0) (Pd₂(dba)₃), copper (I)-thiophene-2-carboxylate (CuTC), and triethyl phosphite (P(OEt)₃) in dimethylformamide (DMF) to form tert-butyl (2S,3S,E)-2-(tert-butyldimethylsilyloxy)-4-oxononadec-5-en-3-ylcarbamate (Compound A3). Compound A3 was formed in 85% yield and an enantiomeric excess (ee) of >99.9%.

### Example 3

### Synthesis of Compound of Formula I

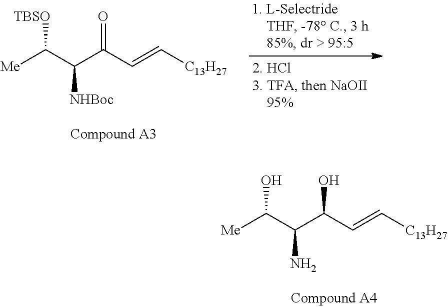

Compound A3 was reduced with a solution of L-Selectride in THF for 3 hours to form tert-butyl (2S,3R,4S,E)-2-(tert-butyldimethylsilyloxy)-4-hydroxynonadec-5-en-3-ylcarbamate (not shown) in 85% yield and a diastereomeric ratio (dr) of greater than 95:5. The resultant compound was then treated with hydrochloric acid (HCl) followed by trifluoroacetic acid (TFA), and then sodium hydroxide (NaOH) to form (2S,3S,4S,E)-3-aminononadec-5-ene-2,4-diol (Compound A4) in 95% yield.

### Example 4

### Synthesis of Compound of Formula VII

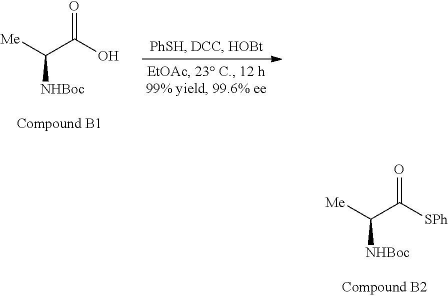

N-Boc-L-alanine (Compound B1) was treated with thiophenol (PhSH), N,N-dicyclohexylcarbodiimide (DCC), and 1-hydroxybenzotriazole (HOBt) in ethyl acetate (EtOAc) to form (S)—S-phenyl 2-(tert-butoxycarbonylamino)propanethioate (Compound B2). Compound B2 was formed in 99% yield and an enantiomeric excess (cc) of 99.6%.

### Example 5

### Synthesis of Compound of Formula VIII

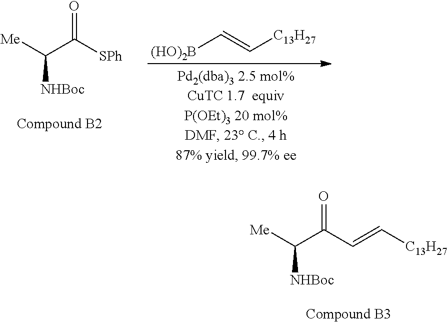

Compound B2 was reacted with (E)-pentadec-1-enylboronic acid in the presence of tris(dibenzylideneacetone)dipalladium(0) (Pd₂(dba)₃), copper (I)-thiophene-2-carboxylate (CuTC), and triethyl phosphite (P(OEt)₃) in dimethylformamide (DMF) to form (S,E)-tert-butyl 3-oxooctadec-4-en-2-ylcarbamate (Compound B3). Compound B3 was formed in 87% yield and an enantiomeric excess (ee) of 99.7%.

### Example 6

### Synthesis of Compound of Formula IX

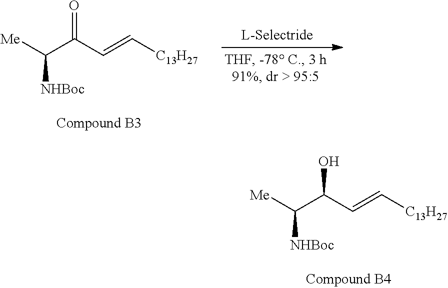

Compound B3 was reduced with a solution of L-Selectride in THF for 3 hours to form tert-butyl (2S,3S,E)-3-hydroxyoctadec-4-en-2-ylcarbamate (Compound B4). Compound B4 was formed in 91% yield and a diastereomeric ratio (dr) of greater than 95:5.

### Example 7

### Synthesis of Compound of Formula X

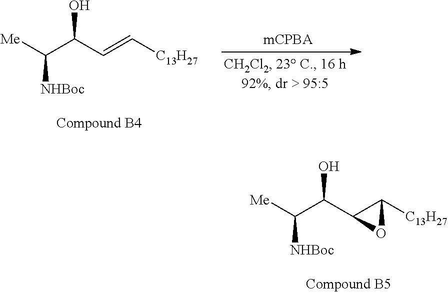

Compound B4 was epoxidized with m-chloroperoxybenzoic acid (mCPBA) in dichloromethane (CH₂Cl₂) to form tert-butyl (1R,2S)-1-hydroxy-1-((2S,3S)-3-tridecyloxiran-2-yl)propan-2-ylcarbamate (Compound B5). Compound B5 was formed in 92% yield and a dr of greater than 95:5.

### Example 8

### Synthesis of Compound of Formula V

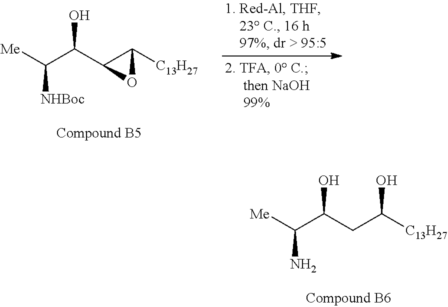

The epoxide ring of Compound B5 was opened by treatment with Red-Al in a solution of THF for 16 hours. The resultant compound (formed in 97%, dr>95:5) was then treated with trifluoroacetic acid (TFA) at 0° C., and then sodium hydroxide (NaOH) to form enigmol (Compound B6) in 99% yield.

A number of embodiments have been described. Nevertheless, it will be understood to one skilled in the art that various modifications may be made. Further, while only certain representative combinations of the methods or products disclosed herein are specifically described, other combinations of the method steps or combinations of elements of a composition or product are intended to fall within the scope of the appended claims. Thus, a combination of steps, elements, or components are included, even though not explicitly stated.

What is claimed is:

1. A method of making a compound of formula (IV): 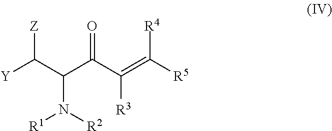
or salt thereof, wherein:
R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl;
R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;
Y is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, substituted or unsubstituted heteroaryl, or substituted or unsubstituted carboxyl; and
Z is H, OH, SH, NR¹R², or substituted or unsubstituted alkyl, with the proviso that when Z is OH, Y is not H;
comprising the step of:
cross-coupling the compound of formula (III) 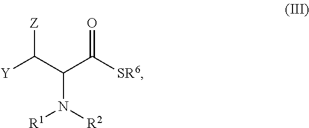
wherein R⁶ is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;
with a compound of the following structure: 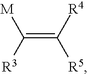
wherein M is B(R⁷)₂, Sn(R⁷)₃, Si(R⁷)₃, ZnR⁷, or InR⁷R⁸, wherein:
R⁷ and R⁸ are each independently selected from halogen, substituted or unsubstituted alkyl, substituted or unsubstituted alkenyl, substituted or unsubstituted alkynyl, substituted or unsubstituted aryl, substituted or unsubstituted alkoxy, substituted or unsubstituted hydroxyl, substituted or unsubstituted heteroalkyl, substituted or unsubstituted heteroalkenyl, substituted or unsubstituted heteroalkynyl, or substituted or unsubstituted heteroaryl;
to form a compound of formula (IV):

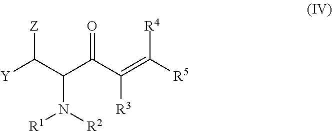

2-10. (canceled)

11. The method of claim 1, wherein Z is OH.

12-63. (canceled)

64. A method of making a compound of formula (I): 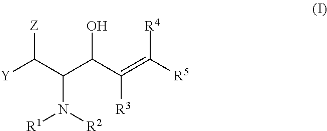
or salt thereof, wherein:
R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl;
R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;
Y is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, substituted or unsubstituted heteroaryl, or substituted or unsubstituted carboxyl; and
Z is H, OH, SH, NR¹R², or substituted or unsubstituted alkyl, with the proviso that when Z is OH, Y is not H;
comprising the step of:
reducing the compound of formula (IV) 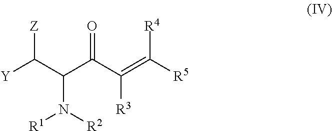
or a pharmaceutically acceptable salt or prodrug thereof, wherein:
R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl;
R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;
Y is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, substituted or unsubstituted heteroaryl, or substituted or unsubstituted carboxyl; and
Z is H, OH, SH, NR¹R², or substituted or unsubstituted alkyl, with the proviso that when Z is OH, Y is not H;
with a reducing agent to form a compound of formula (I).

65. A method of making a compound of formula (VIII): 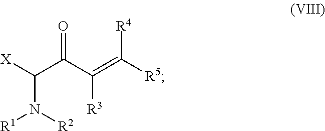
or salt thereof, wherein:
R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl;
R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl; and
X is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;
comprising the step of:
cross-coupling the compound of formula (VII): 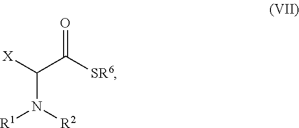
wherein R⁶ is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl;
with a compound of the following formula: 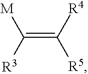
wherein M is B(R⁷)₂, Sn(R⁷)₃, Si(R⁷)₃, ZnR⁷, or InR⁷R⁸, wherein:
R⁷ and R⁸ are each independently selected from halogen, substituted or unsubstituted alkyl, substituted or unsubstituted alkenyl, substituted or unsubstituted alkynyl, substituted or unsubstituted aryl, substituted or unsubstituted alkoxy, substituted or unsubstituted hydroxyl, substituted or unsubstituted heteroalkyl, substituted or unsubstituted heteroalkenyl, substituted or unsubstituted heteroalkynyl, or substituted or unsubstituted heteroaryl;
to form a compound of formula (VIII).

66. The method of claim 64 further comprising the step of reducing the compound of formula (VIII) with a reducing agent to form a compound of formula (IX):

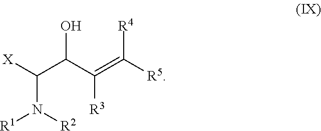

67. The method of claim 65 further comprising the step of directed epoxidation on the compound of formula (IX) to form a compound of formula (X):

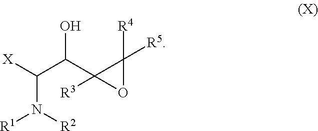

68. The method of claim 66 further comprising the step of opening the epoxide ring of the compound of formula (X) to form a compound of formula (V), 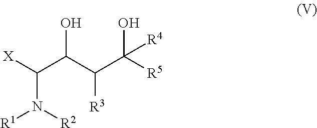
or salt thereof, wherein:
R¹ and R² are each independently selected from H, substituted or unsubstituted alkyl, substituted or unsubstituted alkenyl, substituted or unsubstituted alkynyl, substituted or unsubstituted aryl, substituted or unsubstituted carboxyl, substituted or unsubstituted carbonyl, or substituted or unsubstituted sulfonyl;
R³, R⁴, and R⁵ are each independently selected from H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl; and
X is H, substituted or unsubstituted alkyl, substituted or unsubstituted alkenyl, substituted or unsubstituted alkynyl, substituted or unsubstituted aryl, substituted or unsubstituted heteroalkyl, substituted or unsubstituted heteroalkenyl, substituted or unsubstituted heteroalkynyl, or substituted or unsubstituted heteroaryl.
